(19) United States
(12) Patent Application Publication
(10) Pub. No.: US 2020/0229732 A1
(43) Pub. Date: Jul. 23, 2020

(54) BLOOD VESSEL CONSTRICTION DEVICE

(71) Applicant: Saranas Inc., Houston, TX (US)

(72) Inventors: Mehdi RAZAVI, Houston, TX (US); Zaffer SYED, Houston, TX (US); Kenneth M. BUECHE, Friendswood, TX (US)

(21) Appl. No.: 16/746,536

(22) Filed: Jan. 17, 2020

Related U.S. Application Data
(60) Provisional application No. 62/793,962, filed on Jan. 18, 2019.

(51) Int. Cl. A61B 5/053; A61B 5/02; A61B 5/00; A61M 25/06; A61N 1/05

(52) U.S. Cl. CPC A61B 5/0538; A61B 5/02042; A61N 1/05; A61M 25/0662; A61B 5/6852

(57) ABSTRACT

A vascular access device including a hollow introducer sheath configured to receive a catheter. The sheath is insertable into a blood vessel. The device includes a plurality of electrodes on the introducer sheath, a signal generator coupled to at least two of the plurality of electrodes, and a measurement unit coupled to at least two of the plurality of electrodes. The device further includes a controller coupled to the signal generator and to the measurement unit. The controller is configured to use the measurement unit to obtain impedance values, determine that a bleed is occurring from the impedance values, and in response to the determination that a bleed is occurring, constrict the blood vessel through application of an electrical signal by the signal generator.

### CROSS-REFERENCE TO RELATED APPLICATIONS

This application claims priority to U.S. Provisional Application No. 62/793,962, filed Jan. 18, 2019, which is hereby incorporated by reference.

### BACKGROUND

Bleeding can often be stopped by applying pressure to the wound. However, it may not be readily possible to control internal bleeding through the application of pressure.

### SUMMARY

In one example, a vascular access device including a hollow introducer sheath configured to receive a catheter. The sheath is insertable into a blood vessel. The device includes a plurality of electrodes on the introducer sheath, a signal generator coupled to at least two of the plurality of electrodes, and a measurement unit coupled to at least two of the plurality of electrodes. The device further includes a controller coupled to the signal generator and to the measurement unit. The controller is configured to use the measurement unit to obtain impedance values, determine that a bleed is occurring from the impedance values, and in response to the determination that a bleed is occurring, constrict the blood vessel through application of an electrical signal by the signal generator.

### BRIEF DESCRIPTION OF THE DRAWINGS

For a detailed description of various examples, reference will now be made to the accompanying drawings in which:

FIG. 1 illustrates the use of an impedance monitor and constriction device to detect a bleed and apply an electrical signal to electrodes around the area of the bleed to cause the blood vessel to constrict to thereby stop the bleed.

FIG. 2 shows a method of operation of the impedance monitor and constriction device.

FIG. 3 shows an example implementation of the impedance monitor and constriction device.

### DETAILED DESCRIPTION

As described in U.S. Pat. No. 10,264,981 entitled “Introducer Sheath With Electrodes” and incorporated herein by reference, bioimpedance can be used to detect an internal bleed condition. For example, during a catheter procedure, the blood vessel being accessed may slowly bleed into the surrounding space, and this bleed may not be immediately detected by the medical staff. U.S. Pat. No. 10,264,981 describes an introducer sheath that includes one or more electrodes connected to an electrical circuit. The circuit can determine the impedance (or a value indicative of impedance) between the electrodes. Impedance varies for different body tissues and the impedance of blood is different than the impedance of other tissue types. Thus, if the area around the electrodes begins to fill with blood (due to a bleed), the impedance in that area will change. During the catheter procedure, the device can periodically determine impedance and provide feedback to the medical staff (e.g., an audible and/or visual indicator) if a bleed condition is detected and progressing.

The present disclosure combines the aforementioned impedance-based bleed detection with a vessel constriction capability. The same or different electrodes on the sheath (or other type of vascular access device) can be used to reduce the diameter of the blood vessel (constriction) if a bleed is detected. Sub-millisecond electrical pulses of current between the electrodes and thus through or around the blood vessel can cause the vessel to constrict, reducing the flow of blood, and permitting the body's inherent coagulation process to stop the bleed. In one example, the constriction protocol includes electrical pulse widths between 1 microsecond and 10 milliseconds at a 1 Hz to 10 Hz rate. The pulse amplitude can be 20V or another suitable voltage. In one embodiment, two signal (e.g., positive) and two return (e.g., negative) electrodes are provided on the introducer sheath to be used during the restriction protocol. The electrical signals applied to the signal electrodes may comprise a stream of electrical pulses having an amplitude of 40 V, a pulse width of 10 ms, and a rate of 1 Hz, although other values of any or all of these parameters are possible as well. The pulses can be uniphasic (all of the same polarity) or biphasic (different polarities). The time duration of the vessel constriction protocol is sufficient to let the vessel to seal itself (e.g., 1-10 minutes). Once the vessel constriction protocol ends, the device can revert back to its impedance/bleed detection mode. If a bleed is again detected, the constriction protocol can again be performed by the device. The device may automatically invoke the constriction protocol upon measuring an impedance level outside the normal (no bleed) range.

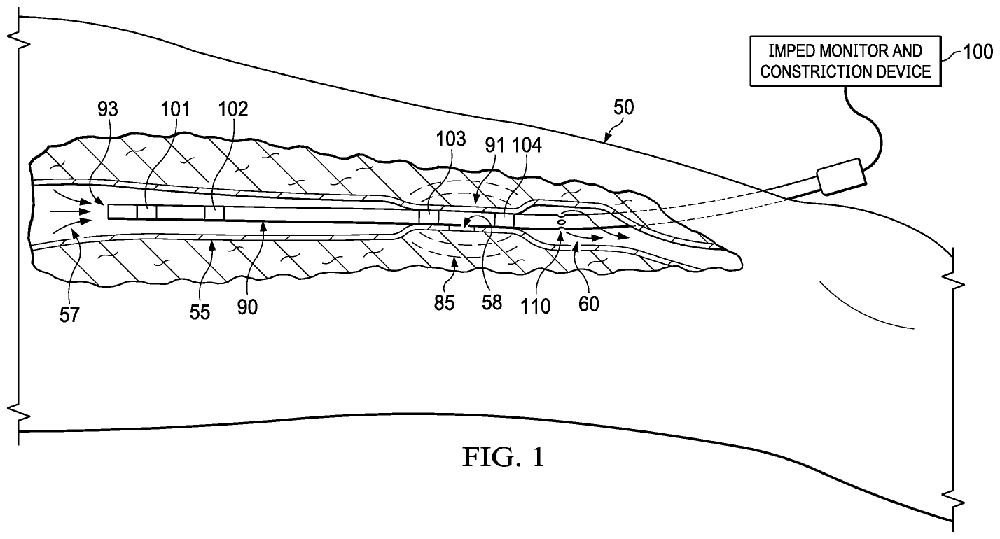

FIG. 1 illustrates an impedance monitor and constriction device 100 usable to temporarily constrict a blood vessel at the location of a bleed to allow the hole in the blood vessel to seal through the body's coagulation process. FIG. 1 illustrates a blood vessel 55 within a person's body (e.g., within a leg 50). The blood vessel 55 has experienced a hole as generally indicated by reference numeral 58. The hole may have been created through a catheterization or other vascular access procedure. As part of the catheterization procedure, an introducer sheath 90 has been inserted into the blood vessel. The introducer sheath 90 is hollow to thereby permit a catheter (not shown) to be inserted therethrough. The direction of blood flow through vessel 55 is indicated at 57 (from left to right in the figure).

The introducer sheath 55 in this example includes multiple electrodes 101, 102, 103, and 104. Although four electrodes 101-104 are shown, a different number of electrodes may be included as desired. The impedance monitor and constriction device 100 is electrically coupled to the electrodes 101-104 via electrical conductors that run along the outside, or within the hollow center, of the introducer sheath 90. Alternatively, the conductors may be embedded within the material comprising the sheath itself.

The impedance monitor and constriction device 100 can measure bioimpedance using the electrodes 101-104 and, from the bioimpedance measurements determine if a bleed is occurring. To measure impedance, the impedance monitor and constriction device 100 may inject a predetermined current magnitude through one pair of electrodes (e.g., electrodes 101 and 104) and measure the resulting voltage using a different set of electrodes (e.g., electrodes 102 and 103). The ratio of voltage to current is impedance. Alternatively, the impedance monitor and constriction device 100 may use one pair of electrodes (e.g., electrodes 101 and 103) to apply a voltage of a predetermined amplitude and measure the resulting current through another pair of electrodes (e.g., electrode 102 and 104). One of the electrodes may be used for both the current injection (or voltage application) as well as the resulting measurement of voltage (or current). The current or voltage applied to the electrodes may be AC or DC. Impedance measurements made at certain frequencies may provide more useful information than at other frequencies. At certain frequencies, it may be difficult to detect a bleed, whereas at other frequencies, bleed detection is easier. In one example, the frequency used for the impedance measurements is in the range of 1000 Hz to 200 KHz, although a different frequency range may be acceptable as well. Additional information regarding impedance measurements may be found in US. Pat. Pub. No. 2017/0049359 which incorporated herein by reference.

The impedance monitor and construction device 100 may compute impedance and compare the computed impedance to threshold values to determine whether a bleed is occurring. For example, during a catherization procedure, an initial impedance measurement may be made, and if subsequent impedance measurements differ from the initial measurement by a predetermined percentage, the impedance monitor and constriction device 100 determines that a bleed is occurring.

If during or after a procedure (e.g., a catheterization), the impedance monitor and constriction device 100 determines that a bleed is occurring, the device 100 can automatically transition to a blood vessel constriction mode of operation in which the device applies an electrical signal of sufficient magnitude to two (or more) of the electrodes 101-104 to cause the blood vessel in the region of the electrodes being energized to constrict. FIG. 1 illustrates a vessel constriction occurring generally at 90 due to electrodes 103 and 104 being used by the impedance monitor and constriction device 100 during the constriction protocol.

During the constriction protocol in which a portion of the blood vessel constricts, the blood vessel is able to self-seal the hole through the body's coagulation process. However, there may be little, or no, blood flow through the constricted part of the blood vessel. FIG. 1 illustrates that one or more perfusion holes 110 may be formed in the introducer sheath downstream from the electrodes. In one example, the combined area of the profusion holes 110 is equal to or greater than the cross-sectional area of the internal diameter of the sheath so as to not cause restriction through the sheath. The perfusion holes 110 permit blood to flow into the sheath 90 through the upstream end (93) of the sheath, through the sheath's internal through bore, through the area of the blood vessel constriction, and out through the perfusion holes 110 to thereby permit blood to continue flowing through the blood vessel and to downstream tissues.

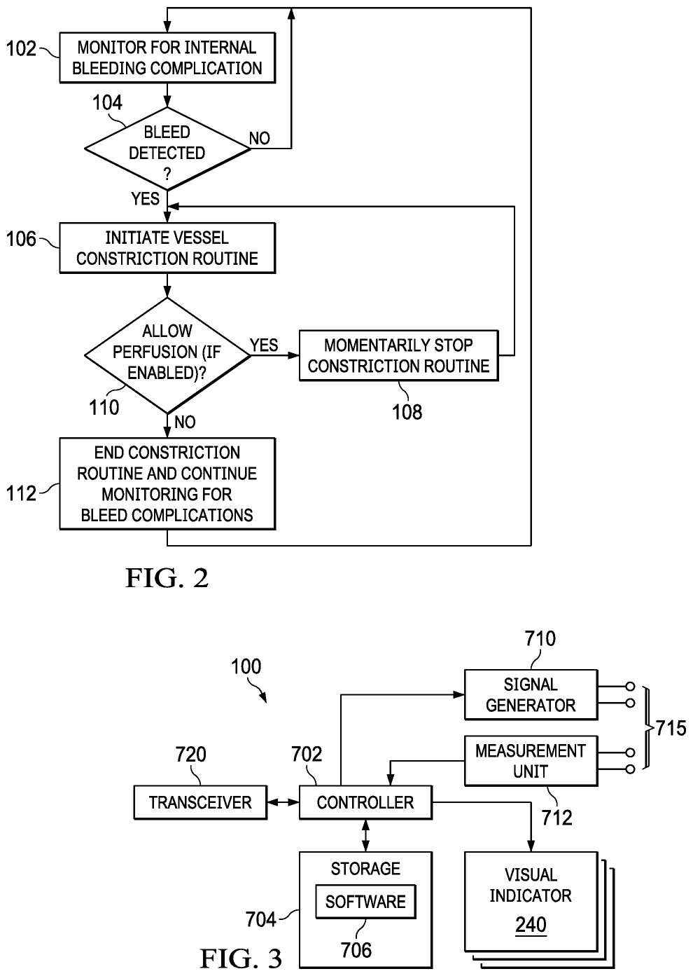

FIG. 2 illustrates a method of operation of the impedance monitor and construction device 100. At 102, the device 100 monitors for a bleed condition by measuring impedance as described above and in U.S. Pat. No. 10,264,981. At 104, if no bleed is detected, control returns to 102 for continued impedance monitoring and bleed detection. Otherwise (if a bleed is detected at 104), the device 100 changes from its bleed detection mode of operation to its vessel constriction mode of operation at 106. As noted above, while the blood vessel is being constricted, little or no blood may flow through past the constriction point (in the example lacking perfusion holes 110). As such, at 110 and 108, the method includes momentarily ceasing the application of signals to the electrodes (if momentary perfusion has been enabled) to momentarily cease the constriction process (110) to allow blood to flow through the blood vessel. Temporary cessation of the constriction protocol may be for a time duration in the range of, for example, 30 seconds to 5 minutes. Once the constriction process ceases (e.g., 15 minutes), the device 100 changes back at 112 from the constriction mode of operation to the bleed detection mode of operation and control loops back to 102. In an embodiment in which the sheath 90 has perfusion holes 110, the device may not perform (may not need to perform) operations 110 and 108).

FIG. 3 shows an example of the impedance monitor and constriction device 100. In this example, the device 100 includes a controller 702, storage 704, visual indicators 240, a signal generator 710, a measurement unit 712, and a transceiver 720. The controller 702 may be a hardware processor that executes software 706 stored in storage 704. Storage 704 may comprise volatile storage (e.g., random access memory) and/or non-volatile storage (e.g., read-only memory). The functionality attributed herein to device 100 may be implemented by the controller 702 upon execution of its software 706. Each visual indicator 240 may comprise a light emitting diode (LED). The visual indicators 240 may be illuminated by controller 702 to indicate the status of the device 100 (e.g., whether a bleed has been detected, whether a vessel constriction protocol has been performed, etc.). The electrodes (e.g. electrode 120, patch electrode 210) are coupled to one or more of the signal generator 710 and measurement unit 720. Upon command by the controller 702, the signal generator 710 generates a predetermined signal (e.g. current or voltage) to be provided to two of the electrodes, and the measurement unit 712 measures the resulting voltage or current as explained above. The controller 702 also may cause the signal generator to generate signals to electrode to constrict a blood vessel as described above. The transceiver 720 may be used to for transmission of impedance values or constriction information (e.g., that a constriction protocol has been invoked for the patient) to an external device (e.g., a bedside monitor). In one example, the transceiver 720 provides a wired interface. In another example, the transceiver 720 provides a wireless interface. An example of a wireless interface includes Bluetooth.

In another embodiment, the impedance monitor and constriction device 100 may be integrated into devices that are placed within the human body via percutaneous access for long term therapeutic modalities, such as in the case of protected percutaneous coronary interventions in which a heart pump (e.g., an Abiomed pump) is used to unload the heart and assist in recovery. In these cases, the pump may be inserted through the femoral artery for up to a month or more where bleeding complications can occur from time to time when the device is moved relative to the patient. In this case the device 100 would continually monitor for changes in the local bioimpedance changes and invoke the constriction routine for a short period of time to reduce the bleed event. Once the bleeding has subsided the constriction routine would be turned off and the device would revert into bleed monitoring mode.

The examples of FIGS. 1 and 2 are directed to embodiments in which device 100 performs both impedance measurement/bleed detection and blood vessel constriction. In other embodiments, a device performs blood vessel constriction but not impedance monitor and bleed detection.

The term “couple” is used throughout the specification. The term may cover connections, communications, or signal paths that enable a functional relationship consistent with the description of the present disclosure. For example, if device A generates a signal to control device B to perform an action, in a first example device A is coupled to device B, or in a second example device A is coupled to device B through intervening component C if intervening component C does not substantially alter the functional relationship between device A and device B such that device B is controlled by device A via the control signal generated by device A.

Modifications are possible in the described embodiments, and other embodiments are possible, within the scope of the claims.

What is claimed is:

1. A vascular access device, comprising:
a hollow introducer sheath configured to receive a catheter, the sheath insertable into a blood vessel;
a first electrode on the introducer sheath;
a second electrode on the introducer sheath;
a signal generator coupled to the first and second electrodes; and
a controller coupled to the signal generator, wherein the controller is configured to constrict the blood vessel through application of an electrical signal by the signal generator to at least one of the first and second electrodes.

2. The vascular access device of claim 1, further including a measurement unit coupled to the controller and to at least one of the first and second electrodes, the measurement unit configured to provide an indication to the controller of an impedance value using the at least one electrode coupled to the measurement unit.

3. The vascular access device of claim 2, wherein the controller is configured to:
cause the measurement unit to obtain an impedance value;
determine, using the impedance value, that a bleed is occurring;
cease causing the measurement unit to obtain an impedance value and instead cause the signal generator to constrict the blood vessel through application of the electrical signal.

4. The vascular access device of claim 3, wherein the controller is further configured to temporarily cease causing the signal generator to constrict the blood vessel to allow perfusion of the blood vessel and then resume causing signal generator to constrict the blood vessel.

5. The vascular access device of claim 4, wherein the controller is further configured to cease causing the signal generator to constrict the blood vessel and again cause the measurement unit to obtain an impedance value.

6. The vascular access device of claim 4, wherein a time duration of the temporary cessation of the signal generator to constrict the blood vessel is in a range of 30 seconds to 5 minutes.

7. The vascular access device of claim 3, wherein the controller is further configured to cease causing the signal generator to constrict the blood vessel and again cause the measurement unit to obtain an impedance value.

8. The vascular access device of claim 7, wherein the introducer sheath includes a perfusion hole downstream from the first and second electrodes, the perfusion hole is configured to allow blood to flow from the inside of the introducer sheath to the outside of the introducer sheath while the signal generator is constricting the blood vessel.

9. The vascular access device of claim 3, wherein the controller is configured to determine that a bleed is occurring through comparison of the impedance value to a threshold.

10. A vascular access device, comprising:
a hollow introducer sheath configured to receive a catheter, the sheath insertable into a blood vessel;
a plurality of electrodes on the introducer sheath;
a signal generator coupled to at least two of the plurality of electrodes;
a measurement unit coupled to at least two of the plurality of electrodes; and
a controller coupled to the signal generator and to the measurement unit, wherein the controller is configured to use the measurement unit to obtain impedance values, determine that a bleed is occurring from the impedance values, and in response to the determination that a bleed is occurring, constrict the blood vessel through application of an electrical signal by the signal generator.

11. The vascular access device of claim 10, wherein the introducer sheath includes a plurality of perfusion holes configured to allow blood to flow from the inside of the introducer sheath to the outside of the introducer sheath while the signal generator is constricting the blood vessel.

12. The vascular access device of claim 10, wherein the controller is further configured to cease causing the signal generator to constrict the blood vessel and again cause the measurement unit to obtain impedance values and to determine whether a bleed is continuing to occur.

13. The vascular access device of claim 10, wherein the controller is further configured to temporarily cease causing the signal generator to constrict the blood vessel to allow perfusion of the blood vessel and then resume causing signal generator to constrict the blood vessel.

14. The vascular access device of claim 3, wherein a time duration of the temporary cessation of the signal generator to constrict the blood vessel is in a range of 30 seconds to 5 minutes.

15. The vascular access device of claim 10, wherein the controller is configured to determine that a bleed is occurring through comparison of the impedance values to a threshold.
